(19) United States
(12) Patent Application Publication
(10) Pub. No.: US 2008/0139842 A1
(43) Pub. Date: Jun. 12, 2008

(54) Medicament for preventive and/or therapeutic treatment of diseases with secretory disorder

(75) Inventors: Koichi SHUDO, Tokyo (JP); Tetsuro MATSUISHI, Saitama (JP); Miwako ISHIDO, Tokyo (JP)

Correspondence Address: GREENBLUM & BERNSTEIN, P.L.C., 1950 ROLAND CLARKE PLACE, RESTON, VA, 20191, US

(73) Assignee: R&R INC., Tokyo (JP)

(21) Appl. No.: 11/854,697

(22) Filed: Sep. 13, 2007

(30) Foreign Application Priority Data
Sep. 27, 2006 (JP) 2006-261845

(51) Int. Cl. C07C 229/00

(52) U.S. Cl. 562/433

(57) ABSTRACT

A medicament for preventive and/or therapeutic treatment of a secretory dysfunctional disease associated with lymphocyte infiltration into a gland such as type I diabetes or Sjogren's syndrome, which comprises as an active ingredient a retinoid such as, for example, 4-[(5,6,7,8-tetrahydro-5,5,8,8-tetramethyl-2-naphthalenyl)carbamoyl]benzoic acid.

### TECHNICAL FIELD

The present invention relates to a medicament for preventive and/or therapeutic treatment of a secretory dysfunctional disease associated with lymphocyte infiltration into glands such as type I diabetes or Sjogren's syndrome.

### BACKGROUND ART

In secretory dysfunctional diseases associated with lymphocyte infiltration into glands, with impairments of endocrine glands or exocrine glands, various kinds of secretions are inhibited by impairments of secretory glands, resulting in symptoms peculiar to each disease. For example, in type I diabetes, β cells in the pancreatic islets of Langerhans, as the pancreatic endocrine glands secreting insulin, are destroyed, resulting in insufficient insulin secretion to cause blood sugar level elevation. The cardinal symptoms include polyuria, polydipsia, loss of weight, severe hunger, diabetic ketoacidosis and the like, and various complications further concur with prolonged hyperglycemia. In Sjoegren's syndrome, as a result of destruction of exocrine glands such as tear gland and salivary gland by lymphocyte infiltration, symptoms of chronic inflammation are developed due to exocrine insufficiency, which are characterized by xerotic pathological conditions. Examples of the cardinal symptoms include dry eye and dry mouth.

In type I diabetes, since blood sugar level control is indispensable for life support of patients, several times per day of insulin administration (usually administered by injection) are required over the whole life. Alternative therapies include pancreas transplantation and pancreatic islet transplantation. However, the transplantations require human donors and use of immunosuppressant over the whole life, and therefore the transplantations are generally performed limitatively for critical patients. At present, no pharmacotherapy has been developed, which can be widely applicable to huge number of type I diabetes patients without complicated procedures of injection and the like. The therapeutic treatments performed for Sjoegren's syndrome are suppression of inflammation and suppression of infiltration of inflammatory cells with adrenocortical steroids and non-steroid anti-inflammatory agents, amelioration of autoimmune responses with immunosuppressants, supplement of tear with artificial tear or eye drops, supplement of saliva with artificial saliva, and the like, and parasympathetic nerve stimulants such as cevimeline hydrochloride are also administered as secretory gland stimulants aiming at increase in bodily secretion. However, these treatments are performed as symptomatic therapies for Sjoegren's syndrome, and any medicament capable of radical treatment for the disease has not yet been developed. Therefore, it is strongly desired to provide a novel medicament having superior effectiveness for the secretory dysfunctional diseases associated with lymphocyte infiltration into glands such as type I diabetes or Sjogren's syndrome.

Retinoic acid (vitamin A acid), an active metabolite of vitamin A, has extremely important physiological functions, e.g., inducing differentiation of immature cells under development processes toward mature cells having specific functions, control of cell proliferation, and life support action. It has been revealed that various vitamin A derivatives synthesized so far also have similar physiological functions, for example, the benzoic acid derivatives disclosed in Japanese Patent Unexamined Publication (KOKAI) Nos. (Sho)61-22047/1986 and (Sho)61-76440/1986, and the compounds described in Journal of Medicinal Chemistry, 1988, Vol. 31, No. 11, p. 2182. “Retinoids” is a general term for retinoic acid and the aforementioned compounds having retinoic acid-like biological activities.

For example, it was proved that all-trans retinoic acid binds as a ligand to the retinoic acid receptor (RAR) present in cellular nucleus, which belongs to the intranuclear receptor super family (Evans, R. M., Science, 240, p. 889, 1988), and regulates proliferation and differentiation of animal cells or cellular mortalities through induction of the activity as a transcription factor (Petkovich, M., et al., Nature, 330, pp. 444-450, 1987). In addition, the existence of retinoid X receptor (RXR) has been elucidated whose ligand is 9-cis-retinoic acid. The retinoid X receptor has been revealed to participate in the expression of the activities of the retinoic acid by inducing or suppressing the transcription of a target gene by forming a homo-dimer or a heterodimer between the retinoic acid receptor (Mangelsdorf, D. J. et al., Nature, 345, pp. 224-229).

It has also been suggested that the aforementioned compounds having the retinoic acid-like biological activities, e.g., 4-[(5,6,7,8-tetrahydro-5,5,8,8-tetramethyl-2-naphthalenyl)carbamoyl]benzoic acid: Am80, also bind to RAR in similar manners to retinoic acid to exhibit their physiological actions (see, Hashimoto, Y., Cell Struct. Funct., 16, pp. 113-123, 1991; Hashimoto, Y., et al., Biochem. Biophys. Res. Commun., 166, pp. 1300-1307, 1990). Clinically, these compounds were found to be useful for therapeutic and preventive treatments of vitamin A deficiency disease, hyperkeratosis of epithelial tissue, rheumatism, delayed allergy, bone diseases, leukemia and certain types of cancer.

As for relation of retinoids and the secretory dysfunctional diseases such as type I diabetes, it is known that the plasma level of vitamin A (retinol), which is one of the retinoids, decreases in type I diabetes patients (Nutrition, 13, pp. 804-806, 1997). Although it is also reported that decrease of the plasma vitamin A level was observed in the streptozotocin-induced rat diabetes model, which is a type I diabetes animal model, it has been revealed that the plasma vitamin A level is not recovered by supply of vitamin A (Br .J. Nutr., 75, pp. 615-622, 1996). It has been reported that diabetes is ameliorated in the BB/W or severely developed diabetes rat, which is a type I diabetes model, when retinol insufficiency is induced by removing retinoids from feed (Metabolism, 45, pp. 248-252, 1996). Therefore, those skilled in the art do not understand that a retinoid exerts any therapeutic effect for type I diabetes.

### DISCLOSURE OF THE INVENTION

An object of the present invention is to provide a medicament that is capable of exhibiting high effectiveness against the secretory dysfunctional diseases associated with lymphocyte infiltration into glands. In particular, the object of the present invention is to provide a medicament that can achieve excellent preventive and/or therapeutic effect against the secretory dysfunctional diseases associated with lymphocyte infiltration into a gland such as type I diabetes or Sjogren's syndrome.

The inventors of the present invention conducted various researches to achieve the foregoing object. As a result, they found that retinoids such as retinoic acid had excellent preventive and/or therapeutic effects against the secretory dysfunctional diseases associated with lymphocyte infiltration into glands such as type I diabetes or Sjogren's syndrome, and thus achieved the present invention.

According to the present invention, provided is a medicament for preventive and/or therapeutic treatment of a secretory dysfunctional disease associated with lymphocyte infiltration into a gland, which comprises a retinoid as an active ingredient.

According to preferred embodiments of the above invention, provided are the aforementioned medicament ,wherein the secretory dysfunctional disease associated with lymphocyte infiltration into a gland is an exocrine disease such as parotitis, sialoadenitis, dacryoadenitis, dry eye syndrome, xerostomia, hidradenitis, or pancreatitis, or an endocrine disease such as hypothyroidism, hypothalamus hypophysitis, hypoparathyroidism, adrenalitis, gonadal inflammation, or insulitis; the aforementioned medicament, wherein the secretory dysfunctional disease associated with lymphocyte infiltration into a gland is epidemic parotitis, acute suppurative parotitis, recurrent parotitis, acute suppurative sialoadenitis, chronic sclerosing sialoadenitis, Sjogren's syndrome, acute suppurative dacryoadenitis, chronic dacryoadenitis, suppurative hidradenitis, chronic pancreatitis, acute pancreatitis, multiple endocrine insufficiency, polyglandular autoimmune syndrome, polyglandular deficiency syndrome, Schmidt syndrome, chronic thyroiditis, autoimmune hypothalamus hypophysitis, autoimmune adrenalitis, Mikulicz syndrome, or type I diabetes; the aforementioned medicament, wherein the secretory dysfunctional disease associated with lymphocyte infiltration into a gland is chronic sclerosing sialoadenitis, Sjogren's syndrome, chronic dacryoadenitis, chronic pancreatitis, multiple endocrine insufficiency, polyglandular autoimmune syndrome, polyglandular deficiency syndrome, Schmidt syndrome, chronic thyroiditis, autoimmune hypothalamus hypophysitis, autoimmune adrenalitis, Mikulicz syndrome, or type I diabetes; and the aforementioned medicament, wherein the secretory dysfunctional disease associated with lymphocyte infiltration into a gland is type I diabetes or Sjogren's syndrome.

According to further preferred embodiments of the above invention, provided are the aforementioned medicament, wherein the retinoid is non-natural retinoid; and the aforementioned medicament, wherein the retinoid has a basic skeleton comprising an aromatic ring bound with an aromatic carboxylic acid or tropolone by means of a bridging group.

According to still further preferred embodiments of the above invention, provided are the aforementioned medicament, wherein the retinoid is capable of binding to retinoic acid receptor (RAR) subtype α and subtype β; the aforementioned medicament, wherein the retinoid is capable of binding to retinoid X receptor X (RXR); the aforementioned medicament, wherein the retinoid has a basic skeleton comprising a substituted phenyl group bound with benzoic acid or tropolone by means of a bridging group; the aforementioned medicament, wherein the retinoid is 4-[(5,6,7,8-tetrahydro-5,5,8,8-tetramethyl-2-naphthalenyl)carbamoyl]benzoic acid or 4-[(3,5-bis-trimethylsilylphenyl)carboxamido]benzoic acid; the aforementioned medicament, wherein the retinoid comprises dibenzo[b,f][1,4]thiazepinylbenzoic acid as a basic skeleton; the aforementioned medicament, wherein the retinoid is 4-[2,3-(2,5-dimethyl-2,5-hexano)dibenzo[b,f][1,4]-thiazepin-11-yl]benzoic acid; and the aforementioned medicament, wherein the retinoid is 4-[5-(4,7-dimethylbenzofuran-2-yl)pyrrol-2-yl]benzoic acid.

From another aspect, provided are use of the above retinoid for manufacture of the aforementioned medicament; and a method for preventive and/or therapeutic treatment of a secretory dysfunctional disease comprising the step of administering an effective amount of the above retinoid to a mammal including a human.

From a further aspect, provided is a medicament for suppressing lymphocyte infiltration into a gland, which comprises a retinoid as an active ingredient.

The medicament of the present invention can exhibit excellent preventive and/or therapeutic effect against the secretory dysfunctional diseases associated with lymphocyte infiltration into a gland such as type I diabetes or Sjogren's syndrome.

### BEST MODE FOR CARRYING OUT THE INVENTION

In the specification, the term “retinoid” means compounds that bind to receptors required for all trans-retinoic acid and 9-cis-retinoic acid to exhibit physiological functions thereof, and thereby exhibit actions similar to those of retinoic acid or a part of the actions, and the term means compounds that have at least one retinoid-like action, for example, one ore more of cell differentiating action, cell proliferation promoting action, life supporting action, and the like. Whether a certain compound is a retinoid or not can be readily determined by the method described in H. de The, A. Dejean, “Retinoids: 10 years on.”, Basel, Karger, 1991, pp. 2-9.

Further, while retinoids generally have a property of binding to a retinoic acid receptor (RAR), and sometimes have property of binding to RXR together with RAR, the retinoid used as the active ingredient of the medicament of the present invention is preferably a retinoid that binds to the subtype α of RAR (RARα) to exhibit an agonistic action. Whether a certain compound is an agonist of RARα or not, also including as for a binding to a retinoic acid receptor subtype, can be readily determined by the method of the aforementioned publication.

As the active ingredient of the medicament of the present invention, any of natural retinoids or non-natural retinoids may be used. Preferably, non-natural retinoid may be used. As the non-natural retinoids, those having a basic skeleton comprising an aromatic ring bound with an aromatic carboxylic acid or tropolone by means of a bridging group may be used.

More specifically, as non-natural retinoids, those represented by the following general formula: B-X-A (wherein B represents an aromatic group which may be substituted, X represents a bridging group, and A represents a carboxylic acid-substituted aromatic group or tropolonyl group) can be used.

As the aromatic group represented by B, a phenyl group which may have a substituent is preferred. Type, number, and substituting position of the substituent on the phenyl group are not particularly limited. As the substituent on the phenyl group, for example, a lower alkyl group can be used (in the specification, the term “lower” means a carbon number of 1 to about 6, preferably 1 to 4). As the lower alkyl group, an alkyl group having a linear or branched chain is preferred, and more specific examples include methyl group, ethyl group, n-propyl group, isopropyl group, n-butyl group, sec-butyl group, tert-butyl group, and the like. Other examples of the substituent on the phenyl group include, for example, a lower alkoxyl group such as methoxy group, a halogen atom (the halogen atom may be any of fluorine atom, chlorine atom, bromine atom, and iodine atom), a lower alkyl-substituted silyl group such as trimethylsilyl group, and the like. As the phenyl group, for example, a phenyl group substituted with 2 to 4 of lower alkyl groups, a phenyl group substituted with 1 or 2 of tri(lower alkyl)silyl group, and the like are preferred, and a phenyl group substituted with 2 to 4 of alkyl groups, a phenyl group substituted with 2 of trimethylsilyl groups, and the like are more preferred.

When two of the lower alkyl groups substituting on the phenyl group are adjacent to each other, they may combine together to form one or two, preferably one of 5- or 6-membered ring together with the ring-constituting carbon atoms of the phenyl group to which they bind. The ring formed as described above may be saturated or unsaturated, and one or more lower alkyl groups such as methyl group and ethyl group may substitute on the ring. On the aforementioned formed ring, preferably 2 to 4 of methyl groups, more preferably 4 of methyl groups, may substitute. For example, it is preferred that two adjacent lower alkyl groups which substitute on the phenyl ring combine together to form 5,6,7,8-tetrahydronaphthalene ring, 5,5,8,8-tetramethyl-5,6,7,8-tetrahydronaphthalene ring, or the like. As the aromatic group represented by B, an aromatic heterocyclic group may also be used. Examples of such retinoid include a retinoid wherein B is a benzofuranyl group which may have a substituent, preferably benzofuran-2-yl group, particularly preferably 4,7-dimethylbenzofuran-2-yl group.

As the carboxylic acid-substituted aromatic group represented by A, a carboxylic acid-substituted phenyl group, a carboxylic acid-substituted heterocyclic group, and the like can be used, and 4-carboxyphenyl group is preferred. Examples of the heterocyclic carboxylic acid constituting the carboxylic acid-substituted heterocyclic group represented by A include, for example, pyrimidine-5-carboxylic acid, and the like. As the tropolonyl group represented by A, tropolon-5-yl group is preferred. On the ring of the carboxylic acid-substituted aromatic group or tropolonyl group, one or more substituents may exist.

Type of the bridging group represented by X is not particularly limited, and examples include, for example, —NHCO—, —CONH—, —N(R^{A})— (R^{A} represents a lower alkyl group, for example, cyclopropylmethyl group and the like), —C(R^{B})(R^{C})— (R^{B} and R^{C} independently represent hydrogen atom, a lower alkyl group, and the like). Further, X may be a divalent aromatic group. For example, X may be pyrrol-diyl group, or the like. Furthermore, the bridging group represented by X and the aromatic group represented by B may combine together to form a ring structure. For example, the basic skeleton of the retinoid represented by B-X-A may be dibenzo[b,f][1,4]thiazepinylbenzoic acid or dibenzo[b,f][1,4]diazepinylbenzoic acid. In the specification, the term “basic skeleton” means a main chemical structure for one or more arbitrary substituents to bind thereto.

As preferred retinoids, all-trans retinoic acid as natural retinoic acid and non-natural retinoid, for example, retinoids comprising a phenyl-substituted carbamoylbenzoic acid or a phenyl-substituted carboxamidobenzoic acid as a basic skeleton can be used. Various retinoids comprising a phenyl-substituted carbamoylbenzoic acid or a phenyl-substituted carboxamidobenzoic acid as a basic skeleton are known. Typical examples of retinoids having a phenyl-substituted carbamoylbenzoic acid as a basic skeleton include Am80 (4-[(5,6,7,8-tetrahydro-5,5,8,8-tetramethyl-2-naphthalenyl)carbamoyl]benzoic acid (refer to Hashimoto, Y., Cell Struct. Funct., 16, pp. 113-123, 1991; Hashimoto, Y., et al., Biochem. Biophys. Res. Commun., 166, pp. 1300-1307, 1990), and typical examples of retinoids having a phenyl-substituted carboxamidobenzoic acid include Tac101 (4-[(3,5-bis-trimethylsilylphenyl)carboxamido]benzoic acid (J. Med. Chem., 33, pp. 1430-1437, 1990).

Preferred retinoids include, for example, compounds represented by the following general formula (I):

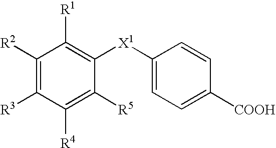

wherein R¹, R², R³, R^{4,} and R⁵ independently represent hydrogen atom, a lower alkyl group, or a lower alkyl-substituted silyl group, when two of adjacent groups among R¹, R², R³, R⁴, and R⁵ are lower alkyl groups, they may combine together to form a 5- or 6-membered ring together with the carbon atoms of the benzene ring to which they bind (this ring may have one or more alkyl groups), and X¹ represents —CONH— or —NHCO—.

In the aforementioned general formula (I), as the lower alkyl group represented by R¹, R², R³, R⁴, and R⁵, a linear or branched alkyl group having 1 to about 6 carbon atoms, preferably 1 to 4 carbon atoms, can be used. For example, methyl group, ethyl group, n-propyl group, isopropyl group, n-butyl group, sec-butyl group, tert-butyl group, and the like can be used. On the aforementioned lower alkyl group, one or more arbitrary substituents may exist. Examples of the substituents include, for example, hydroxyl group, a lower alkoxyl group, a halogen atom, and the like. Examples of the lower alkyl-substituted silyl group represented by R¹, R², R³, R⁴, and R⁵ include, for example, trimethylsilyl group, and the like.

Two of adjacent lower alkyl groups selected from the group consisting of R¹, R², R³, R⁴, and R⁵ may combine together to form one or two, preferably one of 5- or 6-membered ring together with the carbon atoms of the benzene ring to which they bind. The ring formed as described above may be saturated or unsaturated, or an aromatic ring, and one or more lower alkyl groups such as methyl group and ethyl group may substitute on the ring. As the alkyl group which may substitute on the ring, a linear or branched alkyl group having 1 to about 6 carbon atoms, preferably 1 to 4 carbon atoms, can be used. For example, methyl group, ethyl group, and the like can be used, and preferably 2 to 4 of methyl groups, more preferably 4 of methyl groups, may substitute. For example, it is preferred that 5,6,7,8-tetrahydronaphthalene ring, 5,5,8,8-tetramethyl-5,6,7,8-tetrahydronaphthalene ring, or the like is formed by the benzene ring on which R² and R³ substitute, and R² and R³.

Examples of other preferred retinoids include, for example, retinoids comprising dibenzo[b,f][1,4]thiazepinylbenzoic acid or dibenzo[b,f][1,4]-diazepinylbenzoic acid as the basic skeleton represented by B-X-A. Examples of such retinoids are described in, for example, Japanese Patent Unexamined Publication (KOKAI) No. 10-59951. Particularly preferred examples of such retinoids include, for example, HX630 (4-[2,3-(2,5-dimethyl-2,5-hexano)dibenzo[b,f][1,4]-thiazepin-11-yl]benzoic acid). Further, examples of retinoids wherein X is —N(R^{A})—, and B is an aromatic heterocyclic carboxylic acid include, for example, 2-[2-(N-5,6,7,8-tetrahydro-5,5,8,8-tetramethyl-2-naphthalenyl-N-cyclopropylmethyl)amino]pyrimidine-5-carboxylic acid. Further, examples of retinoids wherein X is a divalent aromatic group include, for example, 4-[5-(4,7-dimethylbenzofuran-2-yl)pyrrol-2-yl]benzoic acid. Examples of the compound wherein A is a tropolonyl group include, for example, 5- [[5,6,7,8-tetrahydro-5,5,8,8-tetramethyl-2-naphthalenyl]carboxamido]tropolone, and the like.

Examples of most preferred retinoids include Am80 (4-[(5,6,7,8-tetrahydro-5,5,8,8-tetramethyl-2-naphthalenyl)carbamoyl]benzoic acid) and Am580 (4-[(5,6,7,8-tetrahydro-5,5,8,8-tetramethyl-2-naphthalenyl)carboxamide]benzoic acid).

As the active ingredient of the medicament of the present invention, salts of the above retinoids may be used. For example, physiologically acceptable salts including metal salts such as sodium salts, potassium salts, magnesium salts, and calcium salts, ammonium salts, organic amine salts such as triethylamine salts, and ethanolamine salts, and the like can be used as the active ingredient of the medicament of the present invention. As the active ingredient of the medicament of the present invention, a prodrug of the above retinoid may be used. The term “prodrug” means a compound or a salt thereof which is, after oral or parenteral administration to a mammal, subjected to a structural change such as hydrolysis in vivo, preferably in blood, to produce the above retinoid or a salt thereof. For example, various means for producing prodrugs from pharmaceutical compounds having carboxylic acid, amino group, hydroxyl group or the like are known, and one of ordinary skill in the art can choose appropriate means. Types of the prodrug of the retinoid or a salt thereof are not particularly limited. For example, where a retinoid has carboxylic acid, an example includes a prodrug wherein the carboxylic acid is converted into an alkoxycarbonyl group. Preferred examples include ester compounds such as those formed with methoxycarbonyl group or ethoxycarbonyl group.

The aforementioned retinoid may have one or more asymmetric carbons depending on the types of substituents, and any optical isomers based on these asymmetric carbons, any mixtures of optical isomers, racemates, diastereoisomers based on two or more asymmetric carbons, any mixtures of diastereoisomers, and the like can be used as the active ingredient of the medicament of the present invention. Furthermore, geometrical isomers based on cis- or trans-configuration of double bond, any mixtures of geometrical isomers, and any hydrates or solvates of the compounds in free forms or in the form of a salt can also be used as the active ingredient of the medicament of the present invention.

The medicament of the present invention can be used for preventive and/or therapeutic treatment of a secretory dysfunctional disease associated with lymphocyte infiltration into a gland. In the present specification, “a secretory dysfunctional disease associated with lymphocyte infiltration into a gland” means a disease in which lymphocyte infiltration into exocrine and/or endocrine a gland is observed and whose symptoms include secretory dysfunction. For example, said diseases include exocrine diseases such as parotitis, sialoadenitis, dacryoadenitis, dry eye syndrome, xerostomia, hidradenitis, or pancreatitis, or endocrine diseases such as hypothyroidism, hypothalamus hypophysitis, hypoparathyroidism, adrenalitis, gonadal inflammation, or insulitis. More specifically, said diseases include epidemic parotitis, acute suppurative parotitis, recurrent parotitis, acute suppurative sialoadenitis, chronic sclerosing sialoadenitis, Sjogren's syndrome, acute suppurative dacryoadenitis, chronic dacryoadenitis, suppurative hidradenitis, chronic pancreatitis, acute pancreatitis, multiple endocrine insufficiency, polyglandular autoimmune syndrome, polyglandular deficiency syndrome, Schmidt syndrome, chronic thyroiditis, autoimmune hypothalamus hypophysitis, autoimmune adrenalitis, Mikulicz syndrome, type I diabetes, and the like. Among the secretory dysfunctional diseases associated with lymphocyte infiltration into a gland, chronic sclerosing sialoadenitis, Sjogren's syndrome, chronic dacryoadenitis, chronic pancreatitis, multiple endocrine insufficiency, polyglandular autoimmune syndrome, polyglandular deficiency syndrome, Schmidt syndrome, chronic thyroiditis, autoimmune hypothalamus hypophysitis, autoimmune adrenalitis, Mikulicz syndrome, or type I diabetes is the preferred object to which the medicament of the present invention is applied. Type I diabetes or Sjogren's syndrome is especially preferred disease to which the medicament of the present invention is applied. It can be readily confirmed by those skilled in the art that the medicament of the present invention can exert high effectiveness for the secretory dysfunctional diseases associated with lymphocyte infiltration into a gland by using the method specifically described in the example of the specification.

The medicament of the present invention comprises, as an active ingredient, one or two or more substances selected from the group consisting of the aforementioned retinoid and a salt thereof, and a hydrate thereof and a solvate thereof. A preferred effectiveness may sometimes be obtained by administration of two or more different retinoids in combination. As the medicament of the present invention, the aforementioned substance, per se, may be administered. Preferably, the medicament can be administered as a pharmaceutical composition for oral or parenteral administration which can be prepared by a method well known to one of ordinary skill in the art.

As pharmaceutical compositions suitable for oral administration, examples include tablets, capsules, subtilized granules, granules, liquids, and syrups. As pharmaceutical compositions suitable for parenteral administration, examples include injections, suppositories, inhalant, eye drops, nasal drops, ointments, creams, and plasters. Two or more pharmaceutical compositions may be used in combination. Preferred forms of the medicament of the present invention include pharmaceutical compositions for oral administration.

The aforementioned pharmaceutical composition can be prepared by adding one or two or more kinds of pharmacologically and pharmaceutically acceptable additives for pharmaceutical preparations. Examples of the additives for pharmaceutical preparations include, for example, excipients, disintegrators or disintegrating aids, binders, lubricants, coating agents, dyes, diluents, bases, dissolving agents or dissolving aids, isotonic agents, pH adjusting agents, stabilizing agents, propellants, tackifiers, and the like, but not limited to these.

For example, for preparation of pharmaceutical compositions for oral administration such as tablets, capsules, granules, and powders, excipients such as lactose, crystalline cellulose, and starch, lubricants such as magnesium stearate and talc, binders such as hydroxypropylcellulose and polyvinylpyrrolidone, disintegrators such as carboxymethylcellulose calcium and low substituted hydroxypropylmethylcellulose, coating agents such as hydroxypropylmethylcellulose, macrogol, and silicone resin, and the like may be used as required. For preparation of eye drops, isotonic agents such as sodium chloride, potassium chloride, or concentrated glycerin, buffering agents such as sodium phosphate, sodium acetate, boric acid, or monoethanol amine, stabilizing agents such as sodium citrate or edetate sodium, antiseptics such as benzalkonium chloride and a paraoxybenzoic acid, surface active agents such as polysorbate 80 and polyoxyethylene hydrogenated castor oil, pH adjusting agents such as diluted hydrochloric acid or sodium hydroxide and the like may be used as required. A pH of the eye drop is not particularly limited. A range of 4 to 8 is preferred which is acceptable for phthalic preparations.

A dose of the medicament of the present invention is not particularly limited. The dose may be suitably chosen depending on symptoms, age, body weight and the like of a patient, a method for administration, a type of active ingredient and the like. For example, for oral administrations, a dose of 0.01 to 1,000 mg, preferably 0.1 to 100 mg per day may be administered once or several times as divided portions. However, the aforementioned doses are only for examples, and the dose may be appropriately increased or decreased.

### EXAMPLE

The present invention will be explained more specifically with reference to the example. However, the scope of the present invention is not limited to the following example.

### Example 1

### Evaluation in NOD Mice

NOD mice have been used as spontaneous type I diabetes and Sjoegren's syndrome model animals. Lymphocyte infiltration lesions similar to those of type I diabetes are developed in the pancreatic islets of Langerhans, and lymphocyte infiltration lesions similar to those of Sjoegren's syndrome are developed in the salivary glands (Eur. J. Immunol., 28, pp. 3336-3345, 1998). To female NOD/Shi Jic mice at 6 weeks of age, tamibarotene (Am80, 4-[(5,6,7,8-tetrahydro-5,5,8,8-tetramethyl-2-naphthalenyl)carbamoyl]benzoic acid) was administered over 19 weeks by mixing it in feed. During the test period, urinalysis was conducted once every week to measure urinary sugar. Test paper for urinalyses, Pre Test 3aII (Wako Pure Chemical Industries), was immersed in mouse fresh urine, and when a determined glucose value was 2,000 mg/dl or higher, the mouse was judged to be with diabetes. When the mice gave a sign of physical agony, they were euthanized. After the end of the administration period, pancreases and submaxillary glands were extracted from the euthanized mice, and fixed by immersion in a neutrally buffered 10% formalin fixation solution. From the fixed pancreases and submaxillary glands, hematoxin-eosin stained histopathological specimens were prepared, and the influence of tamibarotene on the pancreases and salivary glands was histopathologically evaluated. For each of the tamibarotene-administered group and the control group, 6 mice were used.

The results are shown in Table 1. The results are indicated by numbers of urinary sugar positive mice, scores for destruction of the pancreatic islets of Langerhans (henceforth occasionally abbreviated as “Pancreatic La islet”) associated with lymphocyte infiltration (Pancreatic La islet lymphocyte infiltration score), scores for decrease in number of the pancreatic islets of Langerhans (Pancreatic La islet number score), and submaxillary gland lymphocyte infiltration scores. The scores were determined according to the following criteria.

### 0: No lesion was observed

### 1: Slight lesion

### 2: Moderate lesions

### 3: Severe Lesions

TABLE 1

| | | | Pancreatic | Submaxillary |
|---|---|---|---|---|
| | Urinary | Pancreatic La | La islet | gland |
| | sugar | islet lymphocyte | number | lymphocyte |
| Administration | positive | infiltration | score | infiltration |
| group (N = 6) | mice | score (average) | (average) | score (average) |
| Control group | 2/6 | 2.7 | 1 | 1.7 |
| 1 mg/kg | 0/6 | 1.7 | 0 | 0.8 |
| Tamibarotene- | | | | |
| administered | | | | |
| group | | | | |

Two of urinary sugar positive mice were observed in the control group, whilst no urinary sugar positive mouse was observed in the tamibarotene-administered group. In the histopathological examination, destruction of pancreatic islets of Langerhans associated with lymphocyte infiltration was observed in the control group. The degree of the destruction was apparently reduced in the tamibarotene-administered group compared with the control group. Also as for the submaxillary glands, expression of lymphocyte infiltration was reduced in the tamibarotene-administered group compared with the control group. From these experimental findings, it was demonstrated that the medicament of the present invention had preventive effect on the onset of type I diabetes with the destruction of the islets of Langerhans by suppressing lymphocyte infiltration in the pancreatic islets of Langerhans and submaxillary gland. Therefore, it was concluded that the medicament of the present invention had effectiveness for preventive and/or therapeutic treatment of the secretory dysfunctional diseases associated with lymphocyte infiltration into a gland.

What is claimed is:

1. A medicament for preventive and/or therapeutic treatment of a secretory dysfunctional disease associated with lymphocyte infiltration into a gland, which comprises a retinoid as an active ingredient.

2. The medicament according to claim 1, wherein the secretory dysfunctional disease associated with lymphocyte infiltration into a gland is an exocrine disease including parotitis, sialoadenitis, dacryoadenitis, dry eye syndrome, xerostomia, hidradenitis, and pancreatitis, or an endocrine disease including hypothyroidism, hypothalamus hypophysitis, hypoparathyroidism, adrenalitis, gonadal inflammation, and insulitis.

3. The medicament according to claim 1, wherein the secretory dysfunctional disease associated with lymphocyte infiltration into a gland is epidemic parotitis, acute suppurative parotitis, recurrent parotitis, acute suppurative sialoadenitis, chronic sclerosing sialoadenitis, Sjogren's syndrome, acute suppurative dacryoadenitis, chronic dacryoadenitis, suppurative hidradenitis, chronic pancreatitis, acute pancreatitis, multiple endocrine insufficiency, polyglandular autoimmune syndrome, polyglandular deficiency syndrome, Schmidt syndrome, chronic thyroiditis, autoimmune hypothalamus hypophysitis, autoimmune adrenalitis, Mikulicz syndrome, or type I diabetes.

4. The medicament according to claim 1, wherein the secretory dysfunctional disease associated with lymphocyte infiltration into a gland is chronic sclerosing sialoadenitis, Sjogren's syndrome, chronic dacryoadenitis, chronic pancreatitis, multiple endocrine insufficiency, polyglandular autoimmune syndrome, polyglandular deficiency syndrome, Schmidt syndrome, chronic thyroiditis, autoimmune hypothalamus hypophysitis, autoimmune adrenalitis, Mikulicz syndrome, or type I diabetes.

5. The medicament according to claim 1, wherein the secretory dysfunctional disease associated with lymphocyte infiltration into a gland is type I diabetes.

6. The medicament according to claim 1, wherein the secretory dysfunctional disease associated with lymphocyte infiltration into a gland is Sjogren's syndrome.

7. The medicament according to claim 1, wherein the retinoid is a non-natural retinoid.

8. The medicament according to claim 6, wherein the retinoid is a retinoid binding to retinoic acid receptor RAR subtype α.

9. The medicament according to claim 6, wherein the retinoid is a retinoid having a basic skeleton comprising an aromatic ring bound with aromatic carboxylic acid or tropolone by means of a bridging group.

10. The medicament according to claim 1, wherein the retinoid is Am80 (4-[(5,6,7,8-tetrahydro-5,5,8,8-tetramethyl-2-naphthalenyl)carbamoyl]benzoic acid) or Am580 (4-[(5,6,7,8-tetrahydro-5,5,8,8-tetramethyl-2-naphthalenyl)carboxamide]benzoic acid).
